(19) United States
(12) Patent Application Publication
(10) Pub. No.: US 2002/0013482 A1
(43) Pub. Date: Jan. 31, 2002

(54) Process for the preparation of dianhydrohexitol bis(4-acryloyloxy)acylates

(75) Inventors: Leonhard Brader, Fischbachau (DE); Norman Haberle, Munchen (DE); Markus Kriegbaum, Munchen (DE)

Correspondence Address: William G. Conger, Brooks & Kushman P.C., 22nd Floor, 1000 Town Center, Southfield, MI, 48075-1351, US

(73) Assignee: Consortium fur elektrochemische Industrie GmbH

(21) Appl. No.: 09/891,950

(22) Filed: Jun. 26, 2001

(30) Foreign Application Priority Data
Jul. 13, 2000 (DE) 100 33 963.8

(51) Int. Cl. C07D 493/04

(52) U.S. Cl. 549/464

(57) ABSTRACT

The invention relates to a process for the preparation of dianhydrohexitol bis(4-acryloyloxy)acylate of the general formula 2

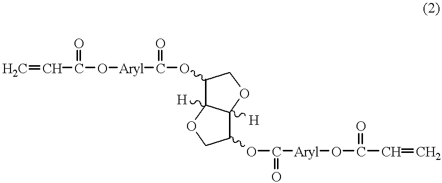

in which, in a first step, dianhydrohexitol bisacylate of the general formula 3

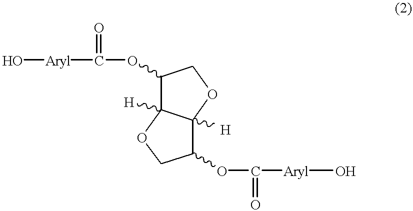

is bisacylated using 3-halopropionyl halide, yielding the corresponding dianhydrohexitol bis[3-halopropionyloxy]arylate, and in which, in a further step, two hydrogen halide molecules are cleaved from the dianhydrohexitol bis[3-halopropionyloxy]arylate with the aid of a base.

### BACKGROUND OF THE INVENTION

1. Field of the Invention

The invention relates to a process for the preparation of dianhydrohexitol bis(4-acryloyloxy)acylates.

2. Background Art

Dianhydrohexitol bis(4-acryloyloxy)acylates are only known in the form of mixtures from which the desired product either cannot be isolated at all, or can be isolated only in highly contaminated form.

If dianhydrohexitols are reacted with acryloyloxy-substituted benzoyl chlorides directly or with the aid of auxiliary bases, various amounts of dianhydrohexitol derivatives of the general formula (1) are formed, depending upon the reaction conditions:

acryloyloxy-(aryl)ₘ-anhydrohexitol-(aryl)ₙ-oxyacryloyl (1)

where m and n are identical or different, but are at least equal to 1, and the sum m+n is greater than 2. These compounds are in the form of mixtures where m+n is greater than 2, and whose individual compounds are difficult to isolate. Owing to their mixed nature, the products are highly viscous or resinous substances which are difficult to process. When use is contemplated as chiral species in liquid-crystalline compounds which require precise and readily reproducible adjustment of absorption wavelengths of pigments produced therewith, such mixtures either cannot be used at all, or produce only highly inferior products.

### SUMMARY OF THE INVENTION

The object of the invention was therefore to provide a process in which dianhydrohexitol bis(4-acryloyloxy)acylates can be obtained in pure form.

### DETAILED DESCRIPTION OF THE PREFERRED EMBODIMENT(S)

The invention relates to a process for the preparation of dianhydrohexitol bis(4-acryloyloxy)acylates of the general formula (2)

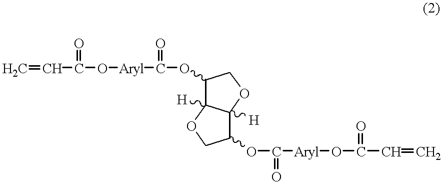

in which “Aryl” is an optionally fluorine-substituted p-phenylene or 2,6-naphthylene radical, in which process, in a first step, dianhydrohexitol bisacylates of the general formula (3)

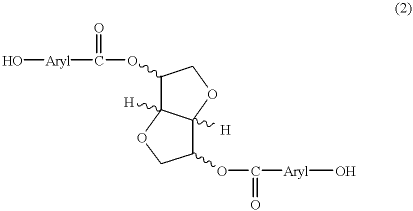

are bisacylated using 3-halopropionyl halide, in which the halogen is chlorine or bromine, yielding the corresponding dianhydrohexitol bis[3-halopropionyloxy]arylate, and in a second step, two hydrogen halide molecules are cleaved from the dianhydrohexitol bis[3-halopropionyloxy]arylate with the aid of a base.

The process gives dianhydrohexitol bis(4-acryloyl)acylates of the general formula (2) in a technically simple manner, inexpensively, in high purity and in good yields. In particular, the process gives few by-products, in particular mixtures of dianhydrohexitol derivatives of the general formula (1). The latter can be reduced to amounts of significantly less than 1%, down to only trace amounts. The pure dianhydrohexitol bis(4-acryloyloxy)acylates of the general formula (2) crystallize well. They are therefore easy to purify and isolate.

In the dianhydrohexitol bis(4-acryloyloxy)acylates of the general formula (2), the wavy lines (bonds) denote freely selectable arrangements of the corresponding chemical bonds above or below the plane of the drawing of the bicyclic dianhydrohexitol system.

Dianhydrohexitol derivatives of the general formula (2) which are suitable for this purpose are isoiditol (1,4;3,6-dianhydro-L-iditol) and isomannide (1,4;3,6-dianhydro-D-mannitol) and, in particular, isosorbide (1,4;3,6-dianhydro-D-glucitol). The central dianhydrohexitol unit is substituted in the 2,5 positions. Particular preference is given to isosorbide 2,5-bis(4-acryloyloxy)benzoate.

The 3-halopropionyl halide employed in the first step is preferably 3-chloropropionyl halide. Preference is furthermore given to 3-halopropionyl chloride, since in the second step, hydrogen halide is cleaved off particularly easily from the corresponding dianhydrohexitol bis[3-bromopropionyloxy]arylates and dianhydrohexitol bis[3-chloropropionyloxy]arylates. 3-Chloropropionyl chloride is particularly preferred.

In the first step, the dianhydrohexitol 2,5-bis(hydroxyarylate) of the general formula (3) and the 3-halopropionyl halide may be introduced into an inert solvent and bisacylated at from 10° C. to 60° C., more preferably at from 20° C. to 40° C., in the presence of base. The dianhydrohexitol 2,5-bis(hydroxyarylate) of the general formula (3) is preferably acylated directly in a first step by slow addition of 3-halopropionyl halide, preferably at from 70° C. to 150° C., more preferably at from 90° C. to 125° C., and preferably at atmospheric pressure, accompanied by thermal expulsion of hydrogen halide gas. The expulsion of the HCl gas and the reaction rate can both be accelerated by passage of an inert gas, such as nitrogen or argon (“stripping”). A variety of combinations of the above process variants are likewise acceptable.

Suitable inert solvents are aromatic hydrocarbons such as benzene, toluene, xylenes or trimethylbenzenes; aliphatic hydrocarbons such as ligroins, “petroleum ethers”, or cyclohexane; open-chain or cyclic ethers such as dibutyl ether, ethylene glycol dimethyl ether, tetrahydrofuran or anisole; esters such as ethyl acetate; ketones, such as acetone or methyl ethyl ketone; and mixtures of these solvents or with other solvents. The solvents and solvent mixtures preferably have a boiling point or boiling range of at most 200° C. at 0.10 mPa.

Non-limiting examples of suitable bases are LiOH, NaOH, KOH and amines. Preferred bases are tertiary amines, in particular triethylamine, tributylamine and pyridine, and mixtures thereof.

The reaction mixture is preferably cooled after a reaction time of from 2 to 10 hours, and any amine hydrochloride formed is filtered off or removed from the reaction mixture by extraction with water. Residues of starting material can be extracted with sodium hydrogencarbonate solution. The dianhydrohexitol 2,5-bis[(3-halopropionyloxy)arylate] product crystallizes, after drying and removal of the solvent by distillation, to give soft, tacky crystals, which may contain a trace of dianhydrohexitol 2,5-bis[(acryloyloxy)acylate] of the general formula (2) and can recrystallized if desired. Suitable solvents for recrystallization are, for example, ethyl acetate, MTB (methyl-tert-butyl ether), and mixtures thereof.

In a further step, the isolated dianhydrohexitol 2,5-bis[(3-halopropionyloxy)arylate] is preferably dissolved in an inert solvent, or alternatively, a purified solution of dianhydrohexitol 2,5-bis[(3-halopropionyloxy)-arylate] or the crude solution of dianhydrohexitol 2,5-bis[(3-halopropionyloxy)arylate] from the first step, is reacted with a base with removal of two molecules of hydrogen halide to give the target end product of the general formula (2). The solvents and bases used in the second step may be the same substances as used in the first step. The removal of hydrogen halide in the second step is preferably carried out at from 40° C. to 100° C., in particular at from 60° C. to 80° C.

In order to stabilize the dianhydrohexitol 2,5-bis(acryloyloxy)acylate of the general formula (2) against premature polymerization, a stabilizer, for example BHDMA (2,6-ditert-butyl-4-(dimethylaminomethylphenol)) is added, preferably before the dehydrohalogenation step.

A preferred variant of the process according to the invention comprises combining the previously described steps in such a way that the reactions are carried out without isolation of the dianhydrohexitol 2,5-bis[(3-halopropionyloxy)arylate]. In this case, the first and second steps occur successively, preferably with an increase in the reaction temperature.

The dianhydrohexitol bisacylates of the general formula (3) employed in the first step can be prepared by esterification from aromatic, hydroxy-substituted carboxylic acids of the general formula (4)

HO—CO-Aryl-OH (4),

by reaction with free dianhydrohexitols, preferably in mixtures of polar and nonpolar solvents, or in nonpolar solvents, and in the presence of a highly acidic catalyst, with removal of water of reaction formed during the reaction.

In a preferred embodiment, the dianhydrohexitol bisacylate of the general formula (3) is prepared as described above, and is not isolated, but instead, as described above, is converted into dianhydrohexitol 2,5-bis[(3-halopropionyloxy)aryl]acylate (in a first step) and, preferably without isolating the latter, into dianhydrohexitol bis(4-acryloyloxy)acylates of the general formula (2) (second step). In particular, the dianhydrohexitol bisacylate of the general formula (3) is employed in the first step as the product suspension formed directly from its preparation.

The reaction can be carried out at atmospheric pressure (0.1 MPa) or, in suitable reaction vessels, for example, autoclaves, at elevated or reduced pressure. It should be noted that all the above symbols in the above formulae are defined independently of one another. In the following examples, unless otherwise stated, all amounts and percentages are by weight, all pressures are 0.10 MPa (abs.) and all temperatures are 20° C.

“HTP” denotes helical twisting power and is a measure of the twisting power of a chiral species [1/μm]. “BHDMA” denotes 2,6-di-tert-butyl-4-(dimethylaminomethyl)phenol, and “MTB” denotes t-butyl methyl ether.

### EXAMPLE 1

This example illustrates the preparation of isosorbide 2,5-bis[4-(3-chloropropionyloxy)benzoate] from isosorbide 2,5-bis(4-hydroxybenzoate).

26.7 g (0.21 mol) of 3-chloropropionyl chloride were added at 110° C. to 38.7 g (0.1 mol) of isosorbide 2,5-bis(4-hydroxybenzoate) in 50 ml of toluene, and the resulting mixture was refluxed for 6 hours with thorough stirring and concurrent elimination of HCl. Toluene was removed by distillation at 90° C. and 1013-1015 mbar, 50 ml of ethyl acetate were added at 60° C., and the product, which precipitates in a glass-like form at 20° C., was agitated with 200 ml of cyclo-hexane. After filtration and drying, the target product, 2,5-bis[4-(3chloropropionyloxy)benzoate, containing 2% of isosorbide 2,5-bis(4-acryloyloxy)benzoate, was obtained in a yield of 49.5 g (87% of theory). The product had a melting point of 96-97° C.

### EXAMPLE 2

This example illustrates the preparation of isosorbide 2,5-bis(4-acryloyloxy)benzoate from isosorbide 2,5-bis[4-(3-chloropropionyloxy)benzoate].

49.5 g (0.087 mol) of the product from Example 1, stabilized with 0.1% of BHDMA and in the form of a partial suspension in 200 ml of ethyl acetate, were initially introduced into a reaction vessel, and 20.2 g (0.20 mol) of triethylamine were slowly added at 70° C. The mixture was subsequently stirred for 3.5 hours. The triethylamine hydrochloride formed was then removed by washing at 60° C. with water, any excess amine was extracted with 3% strength hydrochloric acid, and the mixture was subsequently washed to neutrality with distilled water. After cooling to 15° C., the crystalline product was isolated by filtration. After drying at 60° C. under reduced pressure, the yield was 31.4 g (73% of theory). The product had a melting point of 124° C.

### EXAMPLE 3

This example illustrates the preparation of isosorbide 2,5-bis(4-acryloyloxy)benzoate from isosorbide 2,5-bis(4-hydroxybenzoate) without isolation of intermediates.

38.7 g (0.1 mol) of isosorbide 2,5-bis(4-hydroxybenzoate) were introduced into 200 ml of MTB and stabilized with 0.1% of BHDMA. To this mixture, 25.4 g (0.2 mol) of 3-chloropropionyl chloride dissolved in 100 ml of toluene were added, and 42.2 g (0.42 mol) of triethylamine (dissolved in 100 ml of toluene) were added at 20-25° C. over the course of 1.5 hours. The mixture was then stirred at 56-60° C. for 1.5 hours. The mixture was cooled to 20° C. and washed, first with saturated sodium hydrogencarbonate solution, subsequently with 3% strength hydrochloric acid, and then with water, as described previously, the organic solution was dried using sodium sulfate, and the product isolated by filtration. After the MTB had been removed by distillation at a maximum temperature of 60° C. under reduced pressure, 200 ml of cyclohexane were added, and the product dissolved at 85° C. After cooling to 20° C., the crystallized product was separated by filtration and dried. The yield was 44.8 g (91% of theory), and the HTP was measured as 52 [1/μm].

### EXAMPLE 4

This example illustrates the preparation of isomannide 2,5-bis(4-acryloxyloxy)benzoate from isosorbide 2,5-bis(4-hydroxybenzoate), without isolation of intermediates

6.4 g of 50% strength sulfuric acid and 12 g of diethylene glycol dimethyl ether were added to 36.6 g (0.25 mol) of isomannide and 70 g (0.51 mol) of 4-hydroxybenzoic acid in 200 ml of toluene, and the mixture was then esterified for 17 hours employing a water separator until the expected amount of water had been obtained. 66 g (0.52 mol) of 3-chloropropionyl chloride were added to the product suspension at the reflux temperature over the course of 20 minutes, and HCl was subsequently expelled for 2 hours while passing a gentle stream of nitrogen through the mixture at 110° C. The toluene was distilled off at 80° C. and 50 mbar, and 300 ml of ethyl acetate were added. After stabilization with 0.08 g of BHDMA, 60.6 g (0.6 mol) of triethylamine were added at 30-40° C. over the course of 35 minutes, and the mixture was subsequently stirred at 60° C. for 2.5 hours. After the triethylamine hydrochloride formed had been removed by filtration, the product crystallized from the crude filtrate solution over the course of 16 hours after cooling to 20° C. Following filtration, the crude product was dissolved in 100 ml of ethyl acetate at 60° C., the solution was extracted with 3% strength hydrochloric acid, then with saturated sodium hydrogencarbonate solution and subsequently twice with water, and then dried using sodium sulfate. After cooling, the product was crystallized at 10° C. The product was separated by filtration, washed with methanol and dried.

Yield: 32.2 g, corresponding to 25.9% of theory.

m.p.: 132-134° C.

### EXAMPLE 5

This example illustrates the preparation of isosorbide 2,5-bis[(6-acryloyloxy)naphthoate-2].

3.2 g (6.6 mmol) of isosorbide bis(hydroxynaphthoate) were suspended in 40 ml of ethyl acetate, 1.92 g (15.13 mmol) of 3-chloropropionyl chloride were added, and, after 1 mg of BHDMA had been added, 4.04 g (40 mmol) of triethylamine were added at 75° C. over the course of 10 minutes. After 2 hours at 78° C., and following cooling to 40° C., 10 ml of water were added, insoluble constituents were filtered off, and the organic phase, after separation of water, was washed with 10 ml of sodium hydrogencarbonate solution, then with 3% strength hydrochloric acid, and finally with water. After the solvent had been removed by distillation under reduced pressure at 70° C., the product was recrystallized from glacial acetic acid. The yield was 1.5 g (38.8% of theory). m.p.: 171-172° C. The HTP of the product was determined at 70° C. as being 54 [1/μm].

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A process for the preparation of dianhydrohexitol bis(4-acryloyloxy)acylate of the general formula 2 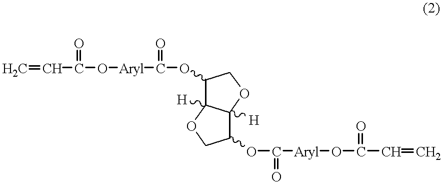
in which Aryl is an optionally fluorine-substituted p-phenylene or 2,6-naphthylene radical, comprising:
in a first step, bisacrylating at least one dianhydrohexitol bisacylate of the general formula (3) 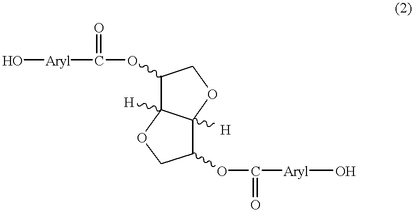
with at least one 3-halopropionyl halide, wherein the halogen of the halo and/or halide of said halopropionyl halide are independently chlorine or bromine, to form a dianhydrohexitol bis[3-halopropionyloxy]arylate, and in a further step or steps, cleaving two hydrogen halide molecules from said dianhydrohexitol bis[3-halopropionyloxy]arylate in the presence of a base.

2. The process of claim 1, wherein the dianhydrohexitol bisacylate of the general formula (3) employed is an isosorbide bisacylate.

3. The process of claim 1, in which the 3-halopropionyl halide employed is 3-chloropropionyl chloride.

4. The process of claim 2, in which the 3-halopropionyl halide employed is 3-chloropropionyl chloride.

5. The process of claim 1, in which the product is isosorbide 2,5-bis(4-acryloyloxy)benzoate.

6. The process of claim 2, in which the product is isosorbide 2,5-bis(4-acryloyloxy)benzoate.

7. The process of claim 3, in which the product is isosorbide 2,5-bis(4-acryloyloxy)benzoate.

8. The process of claim 4, in which the product is isosorbide 2,5-bis(4-acryloyloxy)benzoate.

9. The process of claim 1, in which said first step and said further step are combined in such a way that the respective reactions proceed without isolation of dianhydrohexitol 2,5-bis[(3-halopropionyloxy)arylate].

10. The process of claim 2, in which said first step and said further step are combined in such a way that the respective reactions proceed without isolation of dianhydrohexitol 2,5-bis[(3-halopropionyloxy)arylate].

11. The process of claim 3, in which said first step and said further step are combined in such a way that the respective reactions proceed without isolation of dianhydrohexitol 2,5-bis[(3-halopropionyloxy)arylate].

12. The process of claim 4, in which said first step and said further step are combined in such a way that the respective reactions proceed without isolation of dianhydrohexitol 2,5-bis[(3-halopropionyloxy)arylate].

13. The process of claim 5, in which said first step and said further step are combined in such a way that the respective reactions proceed without isolation of dianhydrohexitol 2,5-bis[(3-halopropionyloxy)arylate].

14. The process of claim 6, in which said first step and said further step are combined in such a way that the respective reactions proceed without isolation of dianhydrohexitol 2,5-bis[(3-halopropionyloxy)arylate].

15. The process of claim 7, in which said first step and said further step are combined in such a way that the respective reactions proceed without isolation of dianhydrohexitol 2,5-bis[(3-halopropionyloxy)arylate].

16. The process of claim 8 in which said first step and said further step are combined in such a way that the respective reactions proceed without isolation of dianhydrohexitol 2,5-bis[(3-halopropionyloxy)arylate].
